(19) United States
(12) Patent Application Publication
(10) Pub. No.: US 2025/0186102 A1
(43) Pub. Date: Jun. 12, 2025

(54) USER INTERFACE MARKING FOR RF THERAPY

(71) Applicant: Boston Scientific Neuromodulation Corporation, Valencia, CA (US)

(72) Inventor: Thien Tich Doan, West Hills, CA (US)

(73) Assignee: Boston Scientific Neuromodulation Corporation, Valencia, CA (US)

(21) Appl. No.: 18/966,575

(22) Filed: Dec. 3, 2024

Related U.S. Application Data
(60) Provisional application No. 63/607,766, filed on Dec. 8, 2023.

(51) Int. Cl. A61B 18/14; A61B 17/00; A61B 18/00

(52) U.S. Cl. CPC A61B 18/14; A61B 2017/00199; A61B 2017/00725; A61B 2018/00339; A61B 2018/00577

(57) ABSTRACT

Radiofrequency ablation systems having a user interface. Pre-therapy planning and testing is performed and data gathered by the radiofrequency ablation system. A user interface for the radiofrequency ablation system displays anatomical mapping locations that have been tested, with indicia of testing outcomes including positive and negative outcome information and the type of testing performed.

### CROSS-REFERENCE TO RELATED APPLICATIONS

This application claims the benefit of U.S. Provisional Patent Application Ser. No. 63/607,766, filed Dec. 8, 2023, which is incorporated herein by reference.

### BACKGROUND

Radiofrequency (RF) ablation is performed in procedures to reduce the passage of pain signals in the body, such as by causing lesions or otherwise reducing the ability of nerves to pass sensory signals. Pulse generators generally do not provide the user (typically a physician) with anatomical information useful for planning and/or executing procedures. For example, probes may be introduced to the region of a target nerve and tested to determine sensory and motor effects induced by electrical signals from such probes. While such testing is routine, the pulse generators, and their graphical user interfaces, provide little, if any, anatomical information to the user. New and alternative interfaces and process flows are desired.

### Overview

The present inventors have recognized, among other things, that a problem to be solved is the need for new and/or alternative tools for helping physicians plan and track activity during an ablation procedure.

A first illustrative and non-limiting example takes the form of a radiofrequency ablation system comprising: a user interface including a screen; a port for coupling to an ablation probe; an input/output circuitry for outputting electrical signals via the port and sensing one or more sensed parameters; a controller; a memory storing controller-readable instructions to perform the following, with a probe coupled to the port and having an electrode at a first electrode location relative to a patient's anatomy: receiving an indication of the first electrode location; generating a first test electrical signal and delivering first test electrical signal to the probe; receiving an indication a patient response to the test electrical signal; and presenting a user an anatomical image on the screen including a first icon indicating each of the first electrode location and the patient response to the first test electrical signal.

Additionally or alternatively, the input/output circuitry is configured to generate each of sensory test electrical signals; motor test electrical signals; and ablation signals; and the first test electrical signal is for a sensory test, and the first icon indicates a sensory test was performed.

Additionally or alternatively, the input/output circuitry is configured to generate each of sensory test electrical signals; motor test electrical signals; and ablation signals; and the first test electrical signal is for a motor test, and the first icon indicates a motor test was performed.

Additionally or alternatively, the controller readable instructions for generating a first test electrical signal and delivering first test electrical signal to the probe include: presenting on the user interface an indication of first test electrical signal parameters, including at least amplitude; receiving from a user an indication to deliver the first test electrical signal; and activating the input/output circuitry to generate and deliver the first test electrical signal using the first test electrical signal parameters.

Additionally or alternatively, the controller readable instructions for receiving an indication of the first electrode location include presenting an anatomy map on the screen, and receiving the indication from the user selecting a position on the screen. Additionally or alternatively, the controller readable instructions for receiving an indication of the first electrode location include receiving imaging information from an imaging system.

Additionally or alternatively, the controller readable instructions for presenting the patient response to the first test electrical signal include instructions to present at least one of a positive sensory result, a negative sensory result, a positive motor result, and a negative motor result, and the first icon is configured to provide a distinct image for each such result. Additionally or alternatively, the controller readable instructions for presenting the patient response to the first test electrical signal include instructions to present a positive sensory result if the patient experiences paresthesia at a desired location. Additionally or alternatively, the controller readable instructions for presenting the patient response to the first test electrical signal include instructions to present a negative sensory result if the patient experiences paresthesia at an undesired location. Additionally or alternatively, the controller readable instructions for presenting the patient response to the first test electrical signal include instructions to present a positive motor result if the patient does not experience a muscle contraction in response to the first test electrical signal. Additionally or alternatively, the controller readable instructions for presenting the patient response to the first test electrical signal include instructions to present a negative motor result if the patient experiences a muscle contraction in response to the first test electrical signal.

Additionally or alternatively, the controller readable instructions further include instructions for: receiving an image of patient anatomy from an imaging system; and presenting the image of patient anatomy on the screen; wherein the controller readable instructions enable the step of receiving an indication of the first electrode location to be performed relative to the image of patient anatomy. Additionally or alternatively, the image of patient anatomy includes at least a portion of a spinal column of a patient.

Additionally or alternatively, the controller-readable instructions further include instructions for receiving an impedance measured during delivery of the first test electrical signal to the probe, comparing the received impedance to a range, determining the received impedance is outside the range, and wherein the first icon indicates the out of range impedance at the first electrode location. Additionally or alternatively, the controller-readable instructions are configured to receive a user selection of the first icon, and display an amplitude and a pulse width of the first test electrical signal.

Another illustrative and non-limiting example takes the form of a method of surgery in a radiofrequency ablation system, the system including a user interface including a screen, a port for coupling to an ablation probe, an input/output circuitry for outputting electrical signals via the port and sensing one or more sensed parameters, and a controller, the method comprising: with a probe coupled to the port and having an electrode at a first electrode location relative to a patient's anatomy, at the user interface, receiving an indication of the first electrode location; with the input/output circuitry, generating and delivering a first test electrical signal to the probe; at the user interface, receiving an indication a patient response to the test electrical signal; and on the user interface, presenting a user an anatomical image including a first icon indicating the first electrode location and the patient response to the first test electrical signal.

Additionally or alternatively, the first test electrical signal is a sensory test electrical signal, and the first icon indicates a sensory test. Additionally or alternatively, the first test electrical signal is a motor test electrical signal, and the first icon indicates a motor test. Additionally or alternatively, the method may include receiving a user selection of the first icon and, in response thereto, presenting to the user on the user interface at least an amplitude of the first test electrical signal. Additionally or alternatively, the anatomical image is an image of a spinal column.

Additionally or alternatively, the method may include receiving imaging information from an imaging system, and the anatomical image is based on the received imaging information. Additionally or alternatively, the method may include presenting on the user interface a second icon indicating a second electrode location and a patient response to a second test electrical signal delivered to the patient at the second electrode location.

Additionally or alternatively, the patient response is selected one of a positive sensory result, a negative sensory result, a positive motor result, and a negative motor result, and the first icon is configured to provide a distinct image for each such result. Additionally or alternatively, the patient response is a positive sensory result when the patient experiences paresthesia at a desired location in response to the first test electrical signal. Additionally or alternatively, the patient response is a negative sensory result when the patient experiences paresthesia at an undesired location. Additionally or alternatively, the patient response is a positive motor result when the patient does not experience a muscle contraction in response to the first test electrical signal. Additionally or alternatively, the patient response is a negative motor result when the patient experiences a muscle contraction in response to the first test electrical signal.

Additionally or alternatively, the method also includes delivering a second test electrical signal to the probe; receiving an impedance measured during delivery of the second test electrical signal to the probe; comparing the received impedance to a range; determining the received impedance is outside the range; and displaying a second icon on the screen indicating the out of range impedance for the second test electrical signal.

Additionally or alternatively, the method also includes receiving a user selection of the first icon; and displaying an amplitude and a pulse width of the first test electrical signal.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate by way of example but not limitation, various embodiments discussed in the present document.

FIG. 1 shows an illustrative RF ablation system and user interface;

FIGS. 2-3 show illustrative process flows for neural RF ablation;

FIG. 4 shows another illustrative RF ablation system;

FIGS. 5-6 show illustrative user interfaces with anatomical maps;

FIG. 7 show an illustrative RF ablation signal generator in block form; and

FIG. 8 shows an illustrative process flow for neural RF ablation.

### DETAILED DESCRIPTION

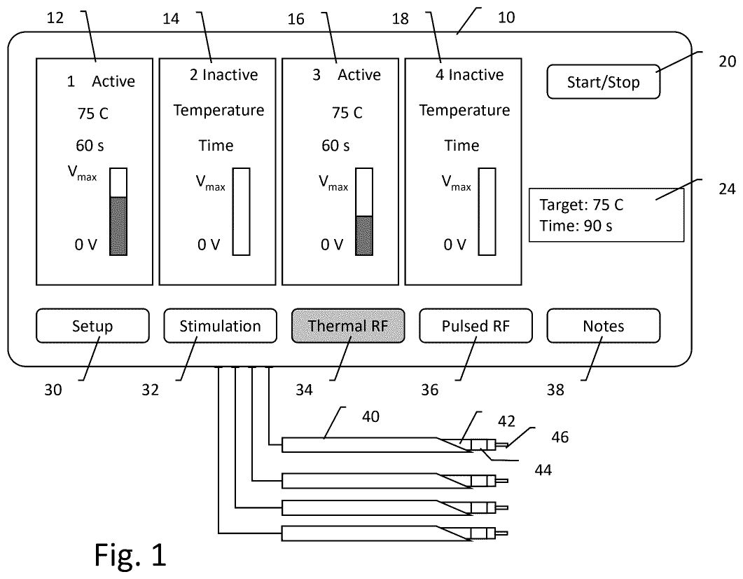

FIG. 1 shows an illustrative RF ablation system and user interface. A signal generator 10 may include a user interface as shown, including separate blocks for a plurality of channels at 12, 14, 16, 18, each illustrating parameters and status for each channel. For example, channel 1, at 12, is “Active”, and data for that channel is shown including the sensed temperature (shown as 75 C), at the probe tip or adjacent the probe electrode, and elapsed time (shown at 60 s). Channels 14 and 18 are indicated as inactive, meaning that probes connected to such channels are not receiving electrical signals at the given time. A voltage bar is shown to indicate, for the given channel, the voltage being delivered to maintain the temperature reading.

A start/stop icon 20 is shown. The user interface as shown may be a touch screen, in whole or in part, if desired. Discrete buttons or dials may replace any of the controls described as icons. System parameters are shown at 24, including, for thermal RF purposes, procedure time and target temperature, for example; other parameters may be shown depending on the selected function at a given time. The parameters displayed in block 24 may be universal so that all channels use the same parameters, or, in the alternative, each individual channel may be controlled using different parameters, as desired. That is, each channel may be independent of each of the other channels in some examples. These parameters in 24 may be adjusted by selecting the setup icon shown at 30, in this example, or by tapping block 24 in other examples. The display shown is for Thermal RF, as indicated by shading at block 34, and other modes may include a Stimulation Mode 32, Pulsed RF Mode 36, and/or a Notes page selectable at 38.

As shown at the bottom of FIG. 1, a plurality of probes may be attached to the signal generator 10. Each probe may have any suitable design. Linear, curved, multi-pronged, sharp or blunt, and other probe designs may be used, for example and without limitation, and common materials include Nitinol and/or stainless steel, though other materials can be used as well. In the example shown, a probe 42 passes through a trocar or sheath 40, with a distally located electrode shown at 44, and a distal tip temperature sensor 46, which may be, for example and without limitation, a thermocouple. While not shown, cooling fluid may flow through the probe 42 and/or trocar 40 to maintain desirable temperature profiles for ablation. For example, cooling fluids may be used to maintain cooler temperatures on the probe 42 itself, and/or on the trocar 40, preventing tissue charring or other overheating effects and providing better controlled and larger lesions, as is known in the art. Various factors can influence lesion size, including, for example and without limitation, time of applied energy or elevated temperature, temperature applied, tip length and/or diameter, selection of monopolar or bipolar signal type, and pre-injection of additional material such as saline, local steroids, etc.

Each of the modes 32, 34, 36 may have different displays and various parameters. A thermal RF mode may use temperature as a setpoint, and may allow temperatures in the range of about 50 C to about 90 C, using output voltages in the range of up to about 150 volts, RMS, using a frequency in the range of 400 kHz to 500 kHz, for example and without limitation. Monopolar or bipolar modes may be available as well. Pulsed RF issues relatively short pulses at generally lower voltages (20 to 100 V RMS, for example), with pulse widths in the range of 1 to 100 milliseconds and pulse repetition rates in the 1 to 20 Hz, for example and without limitation, to achieve temperatures in the range of up to 50 C. RF modes may use any suitable waveshape, for example, sinusoidal. Durations of therapy using thermal RF are generally relatively shorter than for pulsed RF, for example, up to 10 minutes for thermal RF and up to 30 minutes for pulsed RF, for example and without limitation, with typical therapy times generally shorter (1-2 minutes for thermal, and 2-4 minutes for pulsed RF). Other therapy modes and types may be used, as desired, and are known in the art under various trade names.

Generally, thermal RF is used to ablate or destroy tissue, causing localized coagulation and disrupting neural signal paths to interrupt pain signaling. Other therapy forms including pulsed RF are sometimes described as non-destructive and may function by interrupting pain signaling. Non-destructive approaches may be used at the physician's option, for example, if the physician has concerns regarding damage to motor nerves due to proximity, for example.

For motor and/or sensory stimulation, for example, the parameters in block 24 may include a maximum voltage (or maximum current, if a current controlled stimulation is performed), pulse width and frequency, with common ranges for maximum voltage being from about 0.25 volts up to about 7.5 volts, pulse width of 10 to 4000 microseconds, and pulse repetition rate or frequency in the range of about 10 to about 1200 Hz; lesser ranges may be used. For example, one such system has pulse repetition rates of 2 to 200 Hz, pulse width of 100 to 3000 microseconds, voltage of 0 to 5 volts (or current of 0 to 10 milliamps), with voltage controlled or current controlled modes being selectable. Some examples may have different ranges for each of motor and sensory stimulation, while other systems offer the same ranges for both motor and sensory stimulation and allow the physician to make selections for each. These parameters and ranges are not meant to be limiting.

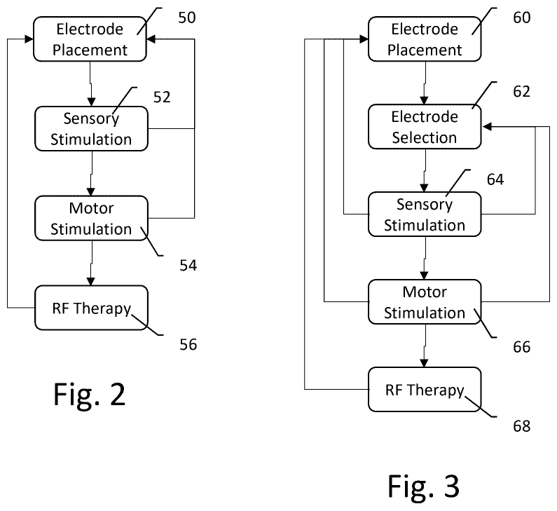

FIGS. 2-3 show illustrative process flows for neural RF ablation. FIG. 2 shows a first example, beginning with electrode placement 50, in which the probes and associated cannulas are placed at target locations. Such placement 50 may be performed with the assistance of fluoroscopic imaging. Even with imaging, the actual proximity to target nerves and non-target nerves can be inexact, and so sensory and/or motor stimulation may be used to confirm desired positioning.

Sensory stimulation 52 generally occurs first, by issuing electrical signals from the electrodes and observing whether the patient experiences paresthesia (often described as a pins-and-needles type sensation) at a desired location. For example, if the patient experiences pathological pain associated with the right hand, the sensory stimulation 52 is delivered with the aim of inducing paresthesia on the right hand, which would indicate that the sensory nerve fiber associated with the right hand has been stimulated and confirms proper placement of the electrode. It is further desirable that sensory stimulation succeed at low amplitudes; if high amplitude is needed, the electrode may be distant from the target nerve and so repositioning may be desired. If sensory stimulation 52 does not confirm proper placement of the electrode, the electrode may be repositioned by cycling back to block 50. Output amplitude is provided first at the lowest setting, generally, and gradually increased until paresthesia is experienced by the patient or an upper limit is reached.

After any needed adjustment steps to achieve desired sensory stimulation 52, motor stimulation takes place at 54. Motor stimulation 54 is performed using different output parameters than sensory stimulation, often with lower frequencies (for example, less than 10 Hz for motor stimulation, and more than 10 Hz for sensory stimulation). If motor stimulation 54 does not cause any motor response (muscle contraction, usually of a limb, may be observed), then the electrode is desirably distant from motor nerves. Again, if motor stimulation 54 elicits a muscle contraction, the system may require electrode repositioning by cycling back to block 50. Output amplitude starts low and is gradually increased until a limit is reached or until a motor response is elicited.

Once the electrode is positioned so that both sensory stimulation 52 and motor stimulation 54 steps are successfully completed with the electrode in that position, ablation can be performed to generate a lesion, or RF treatment may be performed to reduce neural activity of a sensory nerve, as indicated at 56.

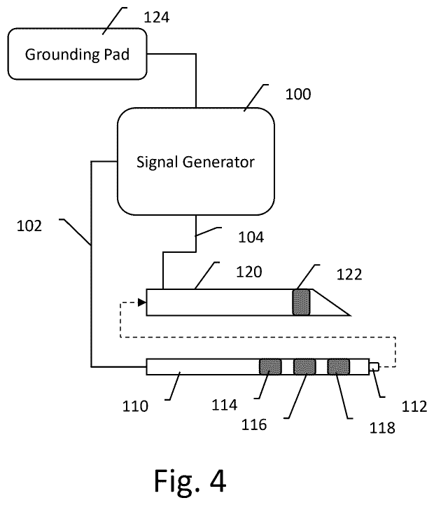

FIG. 3 is similar to FIG. 2, but presents the option of a multi-electrode probe or multi-electrode probe and cannula assembly, examples of which are shown below in FIG. 4. Again, the procedure begins with electrode placement 60, in which a probe and associated cannula are placed under fluoroscopic guidance at a target location. Next, one electrode (for monopolar outputs), or a pair of electrodes (for bipolar outputs), are chosen for testing, at 62. Sensory stimulation 64 is performed as before, as is motor stimulation 66. However, in FIG. 3, the presence of multiple, closely spaced electrodes on the probe means that different electrodes or electrode pairs may be selected at 62 in the event of any failure during sensory stimulation 64 or motor stimulation 66, before repositioning at block 60 is needed. This may help reduce procedure time and offers different options for electrode positioning and combination. Once sensory stimulation 64 and motor stimulation 66 are passed, the RF therapy 68 is performed, using for example, thermal RF, pulsed RF, etc. Multi-electrode probes may also be used to target a longer portion of a nerve by lying in parallel to the nerve and creating a longer lesion along the nerve itself.

FIG. 4 shows another illustrative RF ablation system. Here a signal generator 100 is shown coupled by a first electrical connection 102 to a probe 110. The probe 110 includes a distalmost thermal sensor 112 at its distal tip, and a plurality of electrodes 114, 116, 118 thereon. The probe passes through a cannula 120, which is also connected to the signal generator 100, this time with a second electrical connection 104, and carries an electrode 122 thereon. Each electrode 114, 116, 118, 122 in this system may be separately addressed by pulse generator circuitry in the signal generator 100. Moreover, the probe 110 may be moveable within the cannula 120, allowing adjustment of the position of electrodes 114, 116, 118 relative to electrode 122. This means that the physician can make several positional adjustments without having to remove and reinsert the system, providing an opportunity for finer adjustment of positioning. Fewer or more electrodes may be provided in the probe 110. As with other examples, cooling circulating fluid may be provided to control temperature of the probe body and electrodes themselves during use.

In addition or as an alternative, a grounding pad 124 may be used to allow monopolar therapy to be applied. In a bipolar therapy, two closely spaced electrodes in proximity to the target are used. In monopolar therapy, the current passes between one or more electrodes forming a common pole near the target nerve, and a remote grounding pad 124 which may be placed on the skin of the patient.

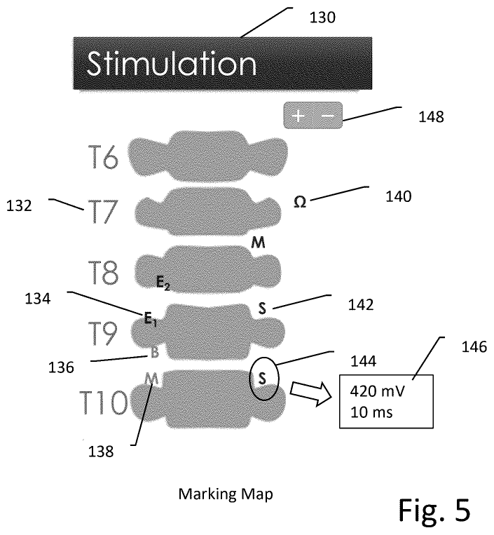

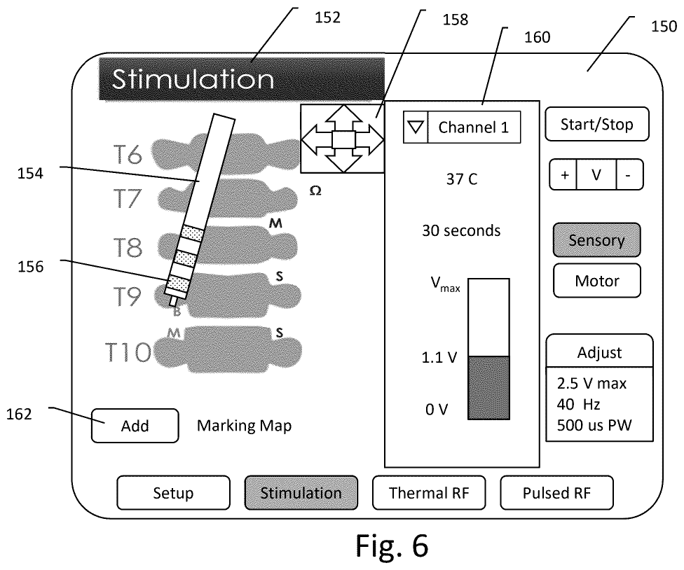

FIGS. 5-6 show illustrative user interfaces with anatomical maps. FIG. 5 shows an anatomical map for use in a stimulation testing process. Alternatively, the mapping may be provided on a fluoroscopy system used during the stimulation testing procedure. The map 130 may be shown on a graphical user interface of an RF signal generator. The map 130 includes a graphical image of the vertebral structures of the spine, and includes level indicia 132 denoting that the thoracic spine is shown (T6 to T10 here). The present location of electrodes is shown at E1 134 and E2 (not marked). This position may be input by a physician, or obtained by the signal generator in communication with an imaging system, such as from the fluoroscopy system. For example, images may be imported showing the patient's spine or other anatomy of interest, and the metal (often nitinol or stainless steel) of the ablation electrodes will show up sharply on such images. Image capture and recognition software may be used, for example, to determine the positions of the bony structures and the electrodes.

Previously tested positions are also noted. Symbol B, shown at 136, may indicate that both sensor and motor testing were performed at the position shown; color coding may be used to indicate whether such testing was successful or failed. Symbol M, shown at 138, indicates motor testing was performed at the position shown; again, color coding may be used, if desired. In other examples, different symbols may be used. At 140, the Greek symbol, Q, appears (the symbol stands for the electrical parameter, ohms), indicating in this example that an impedance test at that location yielded information, such as an out of range impedance (too high, or too low for example). The S shown at 142 indicates a sensory test was performed at that location. The S symbol at 142 may also be color coded for ready recognition of failed or successful test. At 144, another S appears. By tapping the user interface, or selecting using a mouse, trackball, etc., the user can select the symbol at 144 and a pop-up, shown at 146, indicates the voltage and pulse width that caused the sensory response, for example. Each symbol 134, 136, 138, 140, 142, 144 may be referred to as an icon, and is distinct for each of the various patient responses (positive, negative sensory, motor, location and/or impedance) it represents.

In some examples, a “copy/paste” function can be provided to allow the physician/user to select settings from a successful test location to import for use in a new stimulation test, thus importing and/or reusing pulse width and/or amplitude settings. Thus, as the physician performs pre-RF therapy steps, the map 130 allows data to be stored in a readily usable format. If desired, the interface shown can be combined with a live fluoroscopy feed, such as by an overlay, allowing the physician to reposition the electrodes to the desired position, either at or relative to one or more of the symbols. Data may be stored and kept as a patient record for later use if, for example, an additional ablation procedure is performed at a later time.

At 148, an add/remove icon is shown. The user may tap the icon and a pop-up will indicate that by tapping or selecting another location on the screen, a new marker can be placed. When the user taps or selects the new location, the marker type, as well as indication of successor failure at the new location, can then be chosen. The process flow may include allowing the user to use icon 148 to select a new probe/electrode location first, and then to identify the test type and success. Parameters used during a given test may be directly imported from the stimulation controller system, as the pulse width, amplitude and/or any other settings will be already programmed into the system. A zoom-in/out function may be provided as another icon (not shown), or may use the well-known pinch-to-zoom in (and conversely, spread fingers to zoom out) touchscreen function.

FIG. 6 shows another example. Here, the probe itself is shown at 154, with electrodes at 156, on the stimulation mapping 152 which in this case is shown on the graphical user interface 150 (such as a touchscreen) of an RF signal generator. The portion of anatomy shown may be adjusted using the arrows at 158, if desired.

Because testing is typically performed using only one output channel at a time, the user interface here is modified relative to, for example, FIG. 1, showing only the active channel, at 160. A drop-down menu may be used, for example, to allow other channels to be selected. The user interface shows, on the right-hand side, start/stop buttons, a voltage increase/decrease selector, a toggle for sensory or motor testing, and the system parameters in use, which can be adjusted by tapping on “adjust”. Along the bottom of the screen, several different modes can be selected. The user may select the add icon at 162 to add a new marker on the mapping; when a new marker is added, the arrows at 158 may be used to adjust its position, if desired.

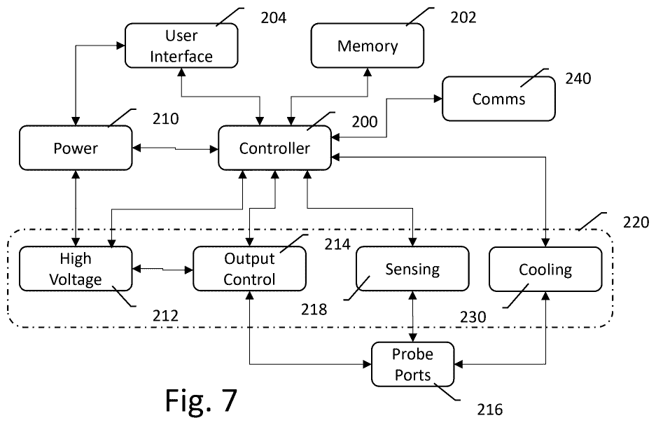

FIG. 7 show an illustrative RF ablation signal generator in block form. The system includes a controller 200. The controller may take many forms, including, for example, a microcontroller or microprocessor, coupled to a memory 202 storing readable instructions for performing methods as described herein, as well as providing configuration of the controller for the various examples that follow. The controller 200 may include one more application-specific integrated circuits (ASIC) to provide additional or specialized functionality, such as, without limitation a signal processing ASIC that can filter received signals from one or more sensors using digital filtering techniques. Logic circuitry, state machines, and discrete or integrated circuit components may be included as well. The skilled person will recognize many different hardware implementations are available for a controller 200.

The controller displays information and options to the user via a user interface 204, which, as noted above, may be a touchscreen, but may also include other items such as buttons, dials, a keyboard, mouse, trackball, etc. A power supply 210, which will typically obtain line electricity, is shown. An input/output circuitry is shown at 220, and may include, for example, a high voltage system 212, an output control 214, and sensing circuitry 218. The output control 214 and sensing circuitry 218 may be coupled to probe ports indicated at 216. For example, the sensing circuitry 218 may sense voltages and/or currents passing through the probe ports 216 to obtain impedance data, and may also communicate with a thermal sensor on the probe. A cooling system 230 is also provided, having therein a cooled fluid circulator (a pump and heat exchanger, for example) that keeps the probe at desired temperatures during ablation. The controller 200 communicates with the components of the input/output circuitry 220 to control operations and obtain data. A communications system 240 is also shown and may use, for example, Bluetooth or other wireless communications mode for communicating with a fluoroscopy system, central server, and/or remote control, if desired.

Various details of the electronics and/or circuitry contained in the system may be similar to or the same as those used in commercially available ablation systems, including for example those from Cosman Instruments, Abbott Medical, etc. Some illustrative examples may also be found in, for example, US PG Pat. Pub. 20230355299, the disclosure of which is incorporated herein by reference, as well as the various patents, literature, and devices, identified or cited therein.

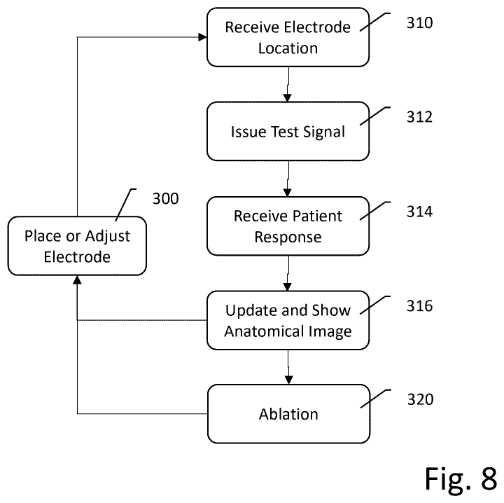

FIG. 8 shows an illustrative process flow for neural RF ablation. As before, the electrode(s) are placed and/or adjusted at 300. The electrode location is received at 310, such as from the physician or by communication with a fluoroscopy system. Test signals (motor or sensory) are generated at 312. The patient response is received at 314, using, for example, data entered by a physician or technician during the testing procedure. The anatomical image is then updated and shown at 316. The electrode may be adjusted at 300, as needed, whether by physically moving the electrode or by selecting a different electrode if more than one is available. When sensory and motor testing are completed, RF therapy (ablation or others, described above) is performed at 320. If more than one therapy site is intended, the process may return to 300.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. The above-described examples (or one or more aspects thereof) may be used in combination with each other. Other embodiments can be used, such as by one of ordinary skill in the art upon reviewing the above description. An Abstract is provided to comply with 37 C.F.R. § 1.72(b), to allow the reader to quickly ascertain the nature of the disclosure and is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of surgery in a radiofrequency ablation system, the system including a user interface including a screen, a port for coupling to an ablation probe, an input/output circuitry for outputting electrical signals via the port and sensing one or more sensed parameters, and a controller, the method comprising:
with a probe coupled to the port and having an electrode at a first electrode location relative to a patient's anatomy,
at the user interface, receiving an indication of the first electrode location;
with the input/output circuitry, generating and delivering a first test electrical signal to the probe;
at the user interface, receiving an indication a patient response to the test electrical signal; and
on the user interface, presenting a user an anatomical image including a first icon indicating the first electrode location and the patient response to the first test electrical signal.

2. The method of claim 1, wherein the first test electrical signal is a sensory test electrical signal, and the first icon indicates a sensory test.

3. The method of claim 1, wherein the first test electrical signal is a motor test electrical signal, and the first icon indicates a motor test.

4. The method of claim 1, further comprising receiving a user selection of the first icon and, in response thereto, presenting to the user on the user interface at least an amplitude of the first test electrical signal.

5. The method of claim 1, wherein the anatomical image is an image of a spinal column.

6. The method of claim 5, further comprising receiving imaging information from an imaging system, and the anatomical image is based on the received imaging information.

7. The method of claim 1, further comprising presenting on the user interface a second icon indicating a second electrode location and a patient response to a second test electrical signal delivered to the patient at the second electrode location.

8. The method of claim 1, wherein the patient response is selected one of a positive sensory result, a negative sensory result, a positive motor result, and a negative motor result, and the first icon is configured to provide a distinct image for each such result.

9. The method of claim 8, wherein the patient response is a positive sensory result when the patient experiences paresthesia at a desired location in response to the first test electrical signal.

10. The method of claim 8, wherein the patient response is a negative sensory result when the patient experiences paresthesia at an undesired location.

11. The method of claim 8, wherein the patient response is a positive motor result when the patient does not experience a muscle contraction in response to the first test electrical signal.

12. The method of claim 8, wherein the patient response is a negative motor result when the patient experiences a muscle contraction in response to the first test electrical signal.

13. The method of claim 1, further comprising:
delivering a second test electrical signal to the probe;
receiving an impedance measured during delivery of the second test electrical signal to the probe;
comparing the received impedance to a range;
determining the received impedance is outside the range; and
displaying a second icon on the screen indicating the out of range impedance for the second test electrical signal.

14. The method of claim 1, further comprising:
receiving a user selection of the first icon; and
displaying an amplitude and a pulse width of the first test electrical signal.

15. A radiofrequency ablation system comprising:
a user interface including a screen;
a port for coupling to an ablation probe;
an input/output circuitry for outputting electrical signals via the port and sensing one or more sensed parameters;
a controller;
a memory storing controller-readable instructions to perform the following, with a probe coupled to the port and having an electrode at a first electrode location relative to a patient's anatomy:
receiving an indication of the first electrode location;
generating a first test electrical signal and delivering first test electrical signal to the probe;
receiving an indication a patient response to the test electrical signal; and
presenting a user an anatomical image on the screen including a first icon indicating each of the first electrode location and the patient response to the first test electrical signal.

16. The radiofrequency ablation system of claim 15, wherein:
the input/output circuitry is configured to generate each of sensory test electrical signals; motor test electrical signals; and ablation signals; and
the first test electrical signal is for a sensory test, and the first icon indicates a sensory test was performed.

17. The radiofrequency ablation system of claim 15, wherein:
the input/output circuitry is configured to generate each of sensory test electrical signals; motor test electrical signals; and ablation signals; and
the first test electrical signal is for a motor test, and the first icon indicates a motor test was performed.

18. The radiofrequency ablation system of claim 15, wherein the controller readable instructions for generating a first test electrical signal and delivering first test electrical signal to the probe include:
presenting on the user interface an indication of first test electrical signal parameters, including at least amplitude;
receiving from a user an indication to deliver the first test electrical signal; and
activating the input/output circuitry to generate and deliver the first test electrical signal using the first test electrical signal parameters.

19. The radiofrequency ablation system of claim 15, wherein the controller readable instructions for receiving an indication of the first electrode location include presenting an anatomy map on the screen, and receiving the indication from the user selecting a position on the screen.

20. The radiofrequency ablation system of claim 15, wherein the controller readable instructions for receiving an indication of the first electrode location include receiving imaging information from an imaging system.
